(19) United States
(12) Plant Patent Application Publication
(10) Pub. No.: US 2003/0079257 P1
(43) Pub. Date: Apr. 24, 2003

(54) Ground cover rose variety 'POULmulti'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/209,634

(22) Filed: Jul. 30, 2002

Related U.S. Application Data
(63) Continuation of application No. 09/268,300, filed on Mar. 16, 1999, now abandoned.

(51) Int. Cl. A01H 5/00

(52) U.S. Cl. PLT/103

(57) ABSTRACT

A new garden rose plant which has attractive foliage and abundant, creamy white, double flowers which are present on the plant almost continually throughout the growing season, and the variety's exceptional disease resistance. This new and distinct variety has shown to be uniform and stable in the resulting generations from asexual propagation.

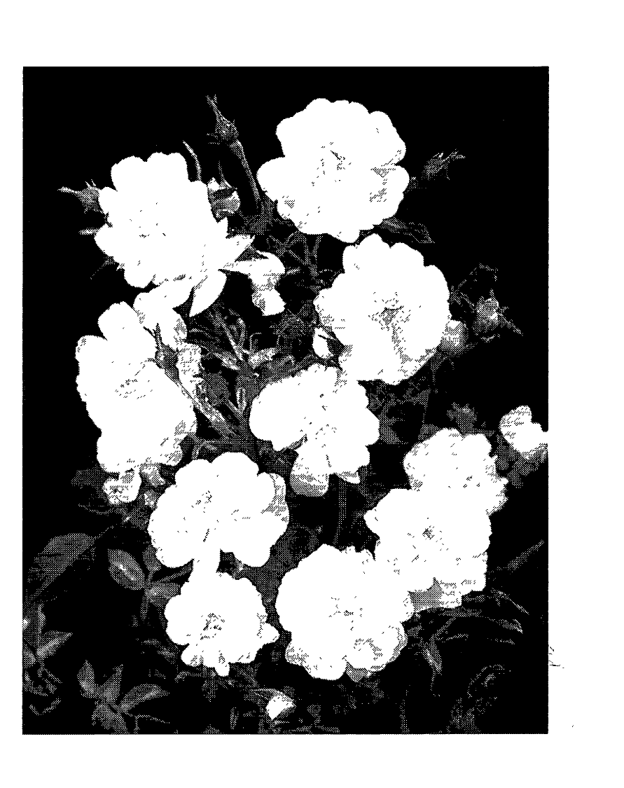

### SUMMARY OF THE INVENTION

The present discovery constitutes a new and distinct variety of a miniature rose plant which was discovered in a cultivated area in Fredensborg, Denmark. The mutation resulted from ‘POULgold’, a non-patented, miniature pot rose hybridized by the same inventors, with PBR Registration Number in Denmark, No. 2108, granted on Jan. 29, 1991. The new rose variety resulted from a naturally occurring mutation of unknown causation on a branch of ‘POULgold’.

The rose plant of the present discovery has a unique combination of characteristics which are outstanding in the new variety and which distinguish it from the original rose ‘POULgold’ as well as all other varieties which we are aware of. For example, the new variety has:

1. Abundant, yellow colored flowers with red intonations on the margins;

2. Vigorous and compact growth,

3. Year-round flowering under glasshouse conditions;

4. Suitability for production from softwood cuttings in pots;

5. Durable flowers and foliage which make the variety suitable for distribution in the floral industry.

The combination of qualities of this variety represents significant improvement over previously available commercial cultivars of this type and distinguishes ‘POULrosit’ from all other varieties of which we are aware.

As part of their rose development program, L. Pemille Olesen and Mogens N. Olesen budded cuttings taken from the aforementioned mutation and conducted evaluations on the resulting seedlings in a controlled environment in Fredensborg, Denmark.

Asexual reproduction of ‘POULrosit’ by cuttings and traditional budding was first done by L. Pernille and Mogens N. Olesen in October of 1998 at their nursery in Fredensborg, Denmark.. This initial and other subsequent propagations conducted in controlled environments have demonstrated that the characteristics of ‘POULrosit’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULrosit’. Specifically illustrated in SHEET 1:

1. Flowering stem showing branching and the attachment of leaves, buds, and peduncles;

2. Flower bud, partially opened bud, and open bloom;

3. Flower petals, detached;

4. Sepals, receptacle, and pedicel;

5. A bare stem with some foliage as well as a bare stem exhibiting thorns;

6. Leaves.

### DETAILED DESCRIPTION OF THE VARIETY

The following is a description of ‘POULrosit’, as observed in its growth in glasshouses in Half Moon Bay, Calif. Color references are made using The Royal Horticultural Society (London, England) Colour Chart, 1995, except where common terms of color are used.

For a comparison, several physical characteristics of the rose variety ‘POULsun’, a miniature rose variety from the same inventors described and illustrated in U.S. Plant Pat. No. 09/590,263 dated Dec. 3, 1996, are compared to ‘POULrosit’ in Chart 1.

CHART 1

| | [POULrosit] | [POULsun] |
|---|---|---|
| Petal Color, | Yellow Group 4A-D with | Yellow Group 13A |
| Upper Surface: | marginal intonations of Red | |
| | Group 52A. | |
| Petal Color, | Yellow Group 4A-C. | Yellow Group 13B |
| Reverse | | |
| Surface: | | |
| Petalage: | Double; 23 to 25 petals. | 30 to 35 |

Parent: Mutation of POULgold

Classification:

Botanical.—Rosa hybrida.

Commercial.—Miniature.

### FLOWER AND FLOWER BUD

Blooming habit: Continuous

Flower bud:

Size.—Upon opening, 15 mm-17 mm in length from base of receptacle to end of bud.

Bud form.—Short, globular.

Bud color.—As sepals unfold Red Group 45B with intonations of Yellow Group 12B, Red Group 45B with intonations of Yellow Group 12B at ¼ opening.

Sepals.—Green Group 139A. Strong foliaceous appendages on three of the five sepals. Surfaces of sepals slightly pubescent. Stipitate glands are present on edges of sepals.

Receptacle.—Surface: Smooth Shape: Cup-shaped. Size: 4 mm (h)×6 mm (w). Color: Yellow-Green Group 144A.

Peduncle.—Surface: Smooth. Length: 25 mm to 30 mm average length. Color: Yellow-Green Group 144A. Strength: Upright.

Borne.—Generally with one to three buds per flowering stem.

Flower bloom:

Fragrance.—None.

Duration.—As a pot plant, flowers last from 7 to 10 days. As a cut flower 4 to 5 days. Petals fall cleanly away from plant.

Size.—Small for a plant grown in a 12.5 cm container, which is typical in production of the variety. Average flower diameter is 35-40 mm when open.

Form.—Shape of flower when viewed from the side: Upon opening, upper part: Convex. Upon opening, lower part: Flat. Open flower, upper part: Flattened convex. Open flower, lower part: Flattened convex.

Petalage.—Average range: 23 to 25 petals under normal conditions, with 2 to 3 petaloids.

Color:

Upon opening petals.—Outermost petals: Upper Surface: Yellow Group 5A. On plants grown under high light conditions margins may exhibit intonations of Red Group 44A. Reverse Side: Yellow Group 5A, On plants grown under high light conditions margins may exhibit intonations of Orange-Red Group 33A. Innermost petals: Upper Surface: Yellow Group 5A. On plants grown under high light conditions margins may exhibit intonations of Red Group 44A. Reverse Side: Yellow Group 5A. On plants grown under high light conditions margins may exhibit intonations of Orange-Red Group 33A.

Upon opening, basal petal spots.—None observed.

After opening, petals.—Outermost petals: Upper Surface: Yellow Group 4A-D, with occasional intonations of Red Group 52A. Reverse Side: Yellow Group 4A-C. Innermost petals: Upper Surface: Yellow Group 4A-D. Reverse Side: Yellow Group 4A-D.

After opening, basal petal spots.—None observed.

General Tonality: On open flower, Yellow Group 4A and 4D. On plants grown under high light conditions, petal margins with intonations of Red Group 52A. No change in the general tonality at the end of the 7th day. Afterwards, general tonality is Yellow-Orange Group 18C.

Petals:

Petal reflex.—Slight.

Petal edge.—Uniform towards center; guard petals are retuse.

Shape.—Deltoid.

Petaloids.—2 to 3 per bloom.

Texture.—Thick.

Arrangement.—Imbricated.

Reproductive Organs:

Pollen.—Color: Yellow-Orange Group 21A. Abundance: Average.

Anthers.—Size: Small. Color: Greyed-Orange Group 177A. Abundance: Very abundant.

Filaments.—Color: Yellow Group 2B.

Stigmas.—Slightly inferior relative to anthers. Color: Yellow-Green Group 145D.

Styles.—Color: Yellow Green Group 145C.

### PLANT

Plant growth: Vigorous, upright to bushy. When grown as a 12.5 cm pot plant, the average height of the plant itself is to 25 cm and the average width is to 20 cm.

Stems:

Color.—Young wood: Yellow-Green Group 144A. Older wood: Yellow-Green Group 144A.

Prickles.—Incidence: Few. Size: Average length: 5 mm. Color: Greyed-Yellow Group 160C. Shape: Linear.

Surface.—Young wood: Smooth. Older wood: Smooth.

Plant foliage: Normal number of leaflets on leaves in middle of the stem: 5 leaflets.

Leaf size.—30 mm (1)×17 mm (w).

Abundance.—Average.

Color.—Upper Leaf Surface: Green Group 139A. Lower Leaf Surface: Green Group 138B. Juvenile foliage: Green Group 138A. Anthocyanin intonation: Occasional. Location: When present, outermost margin of juvenile leaflets. Color: Greyed-Purple Group 184B.

Plant leaves and leaflets:

Stipules.—Size: 4 mm-6 mm. Color: Yellow-Green Group 144A at base; Green Group 139A at tips. Stipitate glands: Present on edges of stipule.

Petiole.—Length: 10 mm-20 mm. Color: Yellow-Green Group 146A. Underneath: Yellow-Green Group 144A. Margins: Few to no stipitate glands present on margins.

Rachis.—Color: Yellow-Green Group 146A. Underneath: Yellow-Green Group 144A; sparse stipitate glands present.

Leaflet.—Edge: Serrated. Shape: Ovate. Texture: Thin and moderately glossy.

Disease resistance: Average resistance to mildew, black spot, and Botrytis under normal growing conditions in Half Moon Bay, Calif.

Cold hardiness: ‘POULrolyt’ has been found to be resistant to damage from cold, heat and drought damage in USDA Zone 7.

1. A new and distinct variety of garden rose plant, substantially as herein illustrated and described as a distinct and novel rose variety due to its abundant, creamy white, double flowers which are present on the plant almost continually throughout the season, and its disease resistance.
